(19) United States
(12) Patent Application Publication
(10) Pub. No.: US 2023/0107794 A1
(43) Pub. Date: Apr. 6, 2023

(54) Multi Chromophore Fluorescent Dyes with Enhanced Brightness

(71) Applicant: Becton, Dickinson and Company, Franklin Lakes, NJ (US)

(72) Inventors: Yongchao Liang, Irvine, CA (US); Yu Chen, San Diego, CA (US)

(21) Appl. No.: 17/850,501

(22) Filed: Jun. 27, 2022

Related U.S. Application Data
(60) Provisional application No. 63/238,989, filed on Aug. 31, 2021.

(51) Int. Cl. G01N 33/58; C09B 67/00

(52) U.S. Cl. CPC G01N 33/582; C09B 68/444

(57) ABSTRACT

Provided are multi-chromophore dyes. Embodiments of the multi-chromophore dyes include two or more dyes each comprising a water solubilizing group, two or more polymeric spacer domains and a linking moiety. Such multi-chromophore dyes can have advantageously high brightness by including multiple dyes while also reducing the rate of self-quenching. Also provided are methods of using such dyes, as well as kits that includes such dyes.

### CROSS-REFERENCE TO RELATED APPLICATION

Pursuant to 35 U.S.C. § 119(e), this application claims priority to the filing dates of U.S. Provisional Patent Application Ser. No. 63/238,989 filed Aug. 31, 2021, the disclosure of which application is incorporated herein by reference in their entirety.

### INTRODUCTION

Fluorescent dyes are compounds which, when irradiated with light of a wavelength which they absorb, emit light of a (usually) different wavelength. Fluorescent dyes find use in a variety of applications in biochemistry, biology and medicine, e.g. in diagnostic kits, in microscopy or in drug screening. Fluorescent dyes are characterized by a number of parameters allowing a user to select a suitable dye depending on the desired purpose. Parameters of interest include the excitation wavelength maximum, the emission wavelength maximum, the Stokes shift, the extinction coefficient, the fluorescence quantum yield and the fluorescence lifetime. Dyes may be selected according to the application of interest in order to, e.g., allow penetration of exciting radiation into biological samples, to minimize background fluorescence and/or to achieve a high signal-to-noise ratio.

Molecular recognition involves the specific binding of two molecules. Molecules which have binding specificity for a target biomolecule find use in a variety of research and diagnostic applications, such as the labeling and separation of analytes, flow cytometry, in situ hybridization, enzyme-linked immunosorbent assays (ELISAs), western blot analysis, magnetic cell separations and chromatography. Target biomolecules may be detected by labeling with a fluorescent dye.

The brightness of a fluorescent dye depends on several factors, such as its molar extinction coefficient and the rate at which the excited-state fluorescent dye is self-quenched. The molar extinction coefficient is also referred to as the molar attentuation coefficient, e.g., as represented by the Greek letter epsilon (ϵ) in the Beer-Lambert law. While individual dye groups can be conjugated to specific binding members like antibodies, such compositions can have low extinction coefficients and therefore low brightness. Although increasing the number of dyes conjugated to a single antibody can raise the extinction coefficient, such modifications can also increase self-quenching, e.g., where dye groups quench one another. Therefore, although the extinction coefficient can be increased by raising the number of dye groups linked to a single antibody, a corresponding increase in quenching can inhibit the overall increase in brightness (where brightness can be quantified as the quantum yield of the fluorescent dye).

### SUMMARY

Provided are multi-chromophore dyes. Embodiments of the multi-chromophore dyes include two or more dyes each comprising water solubilizing groups, two or more polymeric spacer domains and a linking moiety. Such multi-chromophore dyes can have advantageously high brightness by including multiple dyes while also reducing the rate of self-quenching. In some embodiments the polymeric spacer domains are part of a branched backbone joining the dyes to the linking moiety. For example, the multi-chromophore dye can include two, three, or four dyes along with three, four, or five polymeric spacer domains. In some cases, the dyes and polymeric spacer domains are positioned in a linear arrangement as part of the backbone. Poly(alkyene oxide) groups, such as poly(ethylene glycol) groups, are exemplary water soluble groups and polymeric spacer domain groups. Also provided are methods of using such dyes, as well as kits that includes such dyes.

### BRIEF DESCRIPTION OF THE DRAWINGS

FIG. 1 shows the absorption and emission spectra of a multi-chromophore dye according to an embodiment of the invention.

FIG. 2 shows a comparison of brightness and background multi-chromophore dyes according to embodiments of the invention.

### DEFINITIONS

“Alkyl” refers to monoradical, branched or linear, cyclic or non-cyclic, saturated hydrocarbon group. Exemplary alkyl groups include methyl, ethyl, n-apropyl, isopropyl, n-butyl, isobutyl, t-butyl, octyl, decyl, cyclopentyl, and cyclohexyl. In some cases the alkyl group comprises 1 to 24 carbon atoms, such as 1 to 18 carbon atoms or 1 to 12 carbon atoms. The term “lower alkyl” refers to an alkyl groups with 1 to 6 carbon atoms.

“Alkenyl” refers to a monoradical, branched or linear, cyclic or non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl. In some cases the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Alkynyl” refers to a monoradical, branched or linear, cyclic or non-cyclic hydrocarbonyl group that comprises a carbon-carbon triple bond. Exemplary alkynyl groups include ethynyl and n-propynyl. In some cases the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Heterocyclyl” refers to a monoradical, cyclic group that contains a heteroatom (e.g. O, S, N) in as a ring atom and that is not aromatic (i.e.

distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring wherein each of the atoms in the ring are carbon atoms, i.e. none of the ring atoms are heteroatoms (e.g. O, S, N). In some cases the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring wherein at least one of the atoms in the ring is a heteroatom (e.g. O, S, N). Exemplary heteroaryl groups include furyl, thiophenyl, imidazoyl, and pyrimidinyl.

The term “substituted” refers the removal of one or more hydrogens from an atom (e.g. from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no long be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

In addition, the substitutions can themselves be further substituted with one or more groups. For example, the group —C₆H₄CH₂CH₃ can be considered as substituted aryl, i.e. an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein.

Exemplary substituents include alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, alkyl, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR′R″, wherein R′ and R″ may be the same or different and are chosen from hydrogen, optionally substituted alkyl, cycloalkyl, alkenyl, cycloalkenyl, alkynyl, aryl, heteroaryl and heterocyclic.

Diradical groups are also described herein, i.e. in contrast to the monoradical groups such as alkyl and aryl described above. The term “alkylene” refers to the diradical version of an alkyl group, i.e. an alkylene group is a diradical, branched or linear, cyclic or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e. a CHCH₃ fragment where the first atom has two single bonds to other two different groups). The term “arylene” refers to the diradical version of an aryl group, e.g. 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Acyl” refers to a group of formula —C(O)R wherein R is alkyl, alkenyl, or alkynyl. For example, the acetyl group has formula —C(O)CH₃.

“Alkoxy” refers to a group of formula —O(alkyl). Similar groups can be derived from alkenyl, alkynyl, and aryl groups as well.

“Amino” refers to the group —NRR′ wherein R and R′ are independently hydrogen or nonhydrogen substituents, with nonhydrogen substituents including, for example, alkyl, aryl, alkenyl, aralkyl, and substituted variants thereof.

“Halo” and “halogen” refer to the chloro, bromo, fluoro, and iodo groups.

“Carboxyl”, “carboxy”, and “carboxylate” refer to the —CO₂H group and salts thereof.

“Sulfonyl” refers to the group —SO₂R, wherein R is alkyl, alkenyl, alkynyl, aryl, heteroaryl, heterocyclyl, and substituted versions thereof. Exemplary sulfonyl groups include —SO₂CH₃ and —SO₂(C₆H₅).

Unless otherwise specified, reference to an atom is meant to include all isotopes of that atom. For example, reference to H is meant to include ¹H, ²H (i.e., D) and ³H (i.e., T), and reference to C is meant to include ¹²C and all isotopes of carbon (such as ¹³C). In addition, any groups described include all stereoisomers of that group.

### DETAILED DESCRIPTION

Provided are multi-chromophore dyes. Embodiments of the multi-chromophore dyes include two or more dyes each comprising water solubilizing groups, two or more polymeric spacer domains and a linking moiety. Such multi-chromophore dyes can have advantageously high brightness by including multiple dyes while also reducing the rate of self-quenching. In some embodiments the polymeric spacer domais are part of a branched backbone joining the dyes to the linking moiety. For example, the multi-chromophore dye can include two, three, or four dyes along with three, four, or five polymeric spacer domains. In some cases, the dyes and polymeric spacer domains are positioned in a linear arrangement as part of the backbone. Poly(alkyene oxide) groups, such as poly(ethylene glycol) groups, are exemplary water soluble groups and polymeric spacer domain groups. Also provided are methods of using such dyes, as well as kits that includes such dyes.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Multi-Chromophore Dyes

As summarized above, the present disclosure provides multi-chromophore dyes. By multi-chromophore dye is meant a dye that includes two or more, i.e., a pluralty of, distinct chromophore moieties, which chromophore moieties may be the same or different. While the number of distinct chromophore moieties may vary, in some instances the number ranges from 2 to 10, such as 2 to 8, e.g., 2 to 5, including 2 to 3. Multi-chromophore dyes of embodiments of the invention can have advantageously high brightness by including multiple dyes while also reducing the rate of self-quenching, e.g., as compared to a suitable control, such as a dye having multichromophores that is lacks water-solubilizing groups on the chromophore moieties and polymeric spacers domains.

In some cases, the provided multi-chromophore dyes have a brightness that is greater than the brightness of a corresponding reference composition that contains a single dye group or less dye groups than the provided multi-chromophore dye. For example, brightness can be measured as the quantum yield of the number of photons fluorescently emitted divided by the number of photons that contact the sample, e.g., the number of photons absorbed by the multi-chromophore dye. When measuring quantum yield, the sample can be the multi-chromophore dye dissolved in deionized water or in a buffer such as PBS (phosphate buffered saline). For example, the relative brightness of the provided multi-chromophore dye compared to the corresponding reference composition can be 105% or more, such as 110% or more, 115% or more 120% or more, 130% or more, 150% or more, 175% or more, or 200% or more. The provided multi-chromophore dyes can have a higher molar extinction coefficient or higher molar absorptivity than the corresponding reference composition, such as 150% or more, 200% or more, 300% or more, 400% or more, or 500% or more. For example, the molar extinction coefficient can be measured when the multi-chromophore dye is dissolved in deionized water or a buffer such as PBS. The molar extinction coefficient can be measured in any convenient units, such as M⁻¹ cm⁻¹, and can be calculated according to the Beer-Lambert law, which states that absorbance is the product of the molar extinction coefficient, the molar concentration of the multi-chromophore dye, and the distance through which transmitted light travelled (i.e., the path length).

The provided multi-chromophore dyes can also have a rate of self-quenching that is less than a reference multi-chromophore dye. For example, the provided multi-chromophore dye and a reference multi-chromophore dye can have the same number of dyes, but the provided multi-chromophore dye can have a lower rate of self-quenching, e.g., the rate of self quenching is 95% or less compared to the reference multi-chromophore dye, such as 90% or less, 85% or less, 80% or less, 70% or less, 60% or less, 50% or less, 40% or less, 30% or less, 20% or less, or 10% or less. The rate of self quenching can be measured, for example, in deionized water or a buffer such as PBS (phosphate buffered saline). In some cases the reference multi-chromophore dye lacks a polymeric spacer domain, lacks a water soluble group as part of each dye, or a combination thereof. In some cases the polymeric spacer domains, the water solubilizing groups of the dyes, or a combination thereof can cause the reduction in rate of self-quenching.

Multi-chromophore dyes of embodiments of the invention may include two or more dyes each comprising a water solubilizing group, two or more polymeric spacer domains, and a linking moiety. In a given multi-chromophore dye, each dye can have the same or different chemical structure, and each polymeric spacer domain can have the same or different chemical structure, as desired. The components of the dyes are now reviewed in greater detail.

### Dyes

As described above, the multi-chromophore dyes include two or more dyes, each of which include water solubilizing groups of the same or different chemical structure. As used herein, the term “dye” is used interchangeably with the terms “fluorescent dye”, “fluorophore”, “chromophore”, and “dye group”.

Exemplary categories of dyes include rhodamines, perylenes, diimides, coumarins, xanthenes, cyanines, polymethines, pyrenes, thiazines, acridines, dipyrromethene-based dyes, napthalimides, phycobiliproteins, peridinum chlorophyll proteins, and derivatives thereof. Fluorescent dyes of interest include fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY FL, BODIPY FL-Br.sub.2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, Dyonomics dyes (e.g. DY 431, DY 485XL, DY 500XL, DY 610, DY 640, DY 654, DY 682, DY 700, DY 701, DY 704, DY 730, DY 731, DY 732, DY 734, DY 752, DY 778, DY 782, DY 800, DY 831), dipyrromethene borondifluoride (BODIPY), Biotium CF 555, diethylamino coumarin, and derivatives thereof.

In certain cases, the dye is selected from fluorescein, 6-FAM, rhodamine, Texas Red, California Red, iFluor594, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy7®, Cy-Chrome, DyLight 350, DyLight 405, DyLight 488, DyLight 549, DyLight 594, DyLight 633, DyLight 649, DyLight 680, DyLight 750, DyLight 800, phycoerythrin, PerCP (peridinin chlorophyll-a Protein), PerCP-Cy5.5, JOE (6-carboxy-4′,5′-dichloro-2′,7′-dimelhoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, conjugates thereof and combinations thereof.

### Water Solubilizing Groups

As described above, each of the dyes of the multi-chromophore dyes of embodiments of the invention include a water solubilizing group. The dyes can include two or more water solubilizing groups, such as three or more, four or more, five or more, or six or more. The water solubilizing groups can be positioned at any convenient location of the dye.

The term “water solubilizing group” is used interchangeably herein with the terms “water-solubilizing group”, “water soluble group”, and “WSG” to refer to a group that imparts increased water solubility upon the molecule to which it is attached. The increase in water solubility can be assessed by comparing the water solubility of the molecule with the water solubilizing group to a reference molecule that lacks the water solubilizing group, e.g., wherein the water solubilizing group is replaced with a hydrogen atom in the reference molecule. For example, the water solubility can be measured in deionized water or solution containing mostly water, such as a buffer, e.g., PBS (phosphate buffered saline).

In some cases, the water solubilizing group increases the water solubility to more than 2 times the water solubility of the reference compound. Stated in another manner, the water solubility is more than 200% of the water solubility of the reference molecule. In some cases the increased water solubility is more than 5 times, such as more than 10 times, more than 25 times, more than 50 times, or more than 100 times. The water solublilzing group, either alone or in combination with one or more additional water solubilizing groups, can provide solubility in water for the multi-chromophore dye of 10 mg/ml, such as 20 mg/ml or more, 30 mg/ml or more, 40 mg/ml or more, 50 mg/ml or more, 60 mg/ml or more, 70 mg/ml or more, 80 mg/ml or more, 90 mg/ml or more, or 100 mg/ml or more.

A variety of water solublilizing groups can be adapted for use with the provided multi-chromophore dyes to provide for increased water solubility. In some cases, the water solubilizing group is charged, e.g., positively or negatively charged. In some embodiments, the water solubilizing group is zwitterionic, i.e., it has one or more positively charged moities and one or more negatively charged moities, wherein the total negative charge is equal to the total positive charge, causing the water solubilizing group to have no net charge. In some embodiments, the water solubilizing group is neutrally charged and lacks a charged moiety. In some instances, the WSG is linear. In some embodiments, the WSG is branched, e.g., having two, three, four, or more branches.

In some cases the water soluble group includes groups capable of being charged, e.g., by protonation or deprotonation, such as hydroxyl, carboxyl, amine, guanidine, pyridine, pyrazole, imidazole, sulfite, sulfate, sulfinate, sulfonium, phosphite, phosphate, phosphonate, carboxylic anhydrides, and halogen. It is to be understood that the recitation of such groups also includes protonated and deprotonated forms of the groups, e.g., since the acidity or basicity of a solution in which the compositions are dissolved can cause protonation or deprotonation.

In some cases the water soluble group includes moities that are not polymeric and that are not readily charged, but can increase the water solubility of the compositions. For example, moities of interest include halogens (e.g., F, Cl, Br, and I), ethers, esters, aldehydes, and ketones.

In some cases, the WSG is a polymer of repeated hydrophilic monomer units. The term hydrophilic refers to the ability to increase the water solubility of a group. Exemplary polymers that can be used as part of the water solubilizing group include a poly(alkylene oxide), a modified poly(alkylene oxide), polyamide alkylene oxide, a peptide sequence, a peptoid, polyether, polyamines, polyalcohols, a carbohydrate, an oxazoline, a polyol, a dendron, a dendritic polyglycerol, a cellulose, a chitosan, or a derivative thereof. For instance, the polymer can have 2 to 500 repeated hydrophilic monomer units. In some cases the polymers are neutral, i.e., non-ionic. In some cases the polymers are negatively charged, positively charged, or zwitterionic. The term modified polymer, such as a modified PEG, refers to water soluble polymers that have been modified or derivatized at either or both terminals, e.g., to include a terminal substituent (e.g., a terminal alkyl, substituted alkyl, alkoxy or substituted alkoxy, etc.) and/or a terminal linking functional group (e.g., an amino or carboxylic acid group suitable for attachment via amide bond formation) suitable for attached of the polymer to a molecule of interest (e.g., to a light harvesting chromophore via a branching group). It is understood that in some cases, the water soluble polymer can include some dispersity with respect to polymer length, depending on the method of preparation and/or purification of the polymeric starting materials. In some instances, the water soluble polymers are monodisperse.

In some cases the polymer is a polyamide alkylene oxide, such as a polymer having the formula —[C(O)—X—C(O)—NH—Y—NH]ₙ— or —[NH—Y—NH—C(O)—X—C(O)]ₙ—, where X and Y are divalent radicals that may be the same or different and may be branched or linear, and n is an integer from 2-100, such as from 2 to 50, and where either or both of X and Y comprises a biocompatible, substantially non-antigenic water-soluble repeat unit that may be linear or branched.

In some embodiments the WSG is a poly(alkylene oxide) group, e.g., having 2 to 100 alkylene oxide units, such as 3 to 75 units, 4 to 50 units, 5 to 40 units, 6 to 30 units, or 7 to 20 units. The alkylene oxide group can have the formula -(alkyl)-(O)—, wherein the alkyl group can have 2 to 8 carbons, such as 2 to 6 carbons or 2 to 4 carbons. In cases wherein the alkyl group has two carbons, i.e., with the formula —CH₂CH₂—, the poly(alkylene oxide) group is considered as poly(ethylene glycol) group. As such, the poly(ethylene glycol) group has the repeated unit —CH₂CH₂O—.

In some cases, the polyalkylene oxide group, e.g. the polyethylene glycol group, has 2 to 100 alkylene oxide units, such as 3 to 75 units, 4 to 50 units, 5 to 40 units, 6 to 30 units, or 7 to 20 units. In some cases the polyalkylene oxide group is directly connected to another section of the dye, e.g. wherein the another section is selected from rhodamines, perylenes, diimides, coumarins, xanthenes, cyanines, polymethines, pyrenes, thiazines, acridines, dipyrromethene-based dyes, napthalimides, phycobiliproteins, peridinum chlorophyll proteins, and derivatives thereof.

Exemplary polyalkylene oxide groups, such as polyethylene glycol groups, that can be employed as water solubilizing groups are described in “Poly(ethylene glycol) Chemistry: Biotechnical and Biomedical Applications”, J. M. Harris, Ed., Plenum Press, New York, N.Y. (1992); and “Poly(ethylene glycol) Chemistry and Biological Applications”, J. M. Harris and S. Zalipsky, Eds., ACS (1997); and International Patent Applications: WO 90/13540, WO 92/00748, WO 92/16555, WO 94/04193,WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964, and U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966).

The dye can include one or more linkers that connect the water solubilizing group to another section of the dye. For instance, the linker can connect the water solubilizing group to a group selected from a rhodamines, perylenes, diimides, coumarins, xanthenes, cyanines, polymethines, pyrenes, thiazines, acridines, dipyrromethene-based dyes, napthalimides, phycobiliproteins, peridinum chlorophyll proteins, and derivatives thereof. In some embodiments, the water solubilizing group is connected directly to the remainder of the dye without a linker.

In some cases the linker is linear and connects a single water solubilizing group to the remainder of the dye. Exemplary linear linkers include aliphatic moities, such as alkyl groups, alkenyl groups, and alkynyl groups, along with diamino and or diacid units, natural or unnatural amino acids or derivatives, aryl groups, heteroaryl groups, and alkoxy groups, and substituted versions of such groups. In some cases, the linear linker is an alkyl group or a substituted alkyl group.

In some embodiments, the linker is branched and connects two or more water soluble groups to another section of the dye. By using a branched linker, multiple WSGs can be attached at a single location, providing for a greater increase in water solubility compared to a linear linker. For example, the linker can include a cyclic group, e.g., an aryl group or a heteroaryl group, that is substituted with two or more water solubilizing groups. Examples of branched linkers that connect to multiple water soluble groups, e.g., polyethylene glycol groups, are described in U.S. Pat. No. 10,533,092, which is incorporated herein by reference. In some cases, the branched linker is an aryl or heteroaryl group substituted with two, three, four, or five water soluble groups. In certain embodiments, the branching linker is an amino acid, e.g., a lysine amino acid that is connected to three groups via the amino and carboxylic acid groups.

### Polymeric Spacer Domains

The term “polymeric spacer domain” refers to a group that contains a polymeric unit and connects two or more groups, such as connecting a dye to another section of the multi-chromophore dye or connecting a linking group to another section of the multi-chromophore dye. The polymeric unit of the polymeric spacer domain has two or more repeated units.

In some embodiments the repeated units of the polymeric spacer domain are non-conjugated. By “non-conjugated” is meant that at least a portion of the repeat unit includes a saturated backbone group (e.g., a group having two or more consecutive single covalent bonds) which precludes pi conjugation or an extended delocalized electronic structure along the polymeric spacer domain. Exemplary non-conjugated polymeric spacer domains include poly(alkylene oxides), polyamide alkylene oxide, a peptide sequence, a peptoid, polyether, polyamines, polyalcohols, a carbohydrate, an oxazoline, a polyol, a dendron, a dendritic polyglycerol, a cellulose, a chitosan, or a derivative thereof. In some cases the polymeric spacer domain is a water solubilizing group.

In some cases the polymeric spacer domain is a polyamide alkylene oxide, such as a polymer having the formula —[C(O)—X—C(O)—NH—Y—NH]ₙ— or —[NH—Y—NH—C(O)—X—C(O)]ₙ—, where X and Y are divalent radicals that may be the same or different and may be branched or linear, and n is an integer from 2-100, such as from 2 to 50, and where either or both of X and Y comprises a biocompatible, substantially non-antigenic water-soluble repeat unit that may be linear or branched.

In some embodiments the polymeric spacer domain is a poly(alkylene oxide) group. In some embodiments the poly(alkylene oxide) group has 4 to 50 alkylene oxide units, such as 6 to 40 units, 8 to 30 units, 10 to 25 units, or 12 to 20 units. In cases wherein the alkyl group of the alkylene oxide has two carbons, i.e., with the formula —CH₂CH₂—, the poly(alkylene oxide) group is considered as poly(ethylene glycol) group. As such, the poly(ethylene glycol) group has the repeated unit —CH₂CH₂O—.

In some cases there are other groups that connect a dye to a polymeric spacer domain, connect the linking moiety to a polymeric spacer domain, connect one polymeric spacer domain to another polymeric spacer domain, or a combination thereof. Such connecting groups can be, for example, alkyl groups, substituted alkyl groups, aryl groups, substituted aryl groups, heteroaryl groups, or substituted heteroaryl groups. In some cases such connecting groups include one or more amide moieties. In some cases the branched backbone of the multi-chromophore dye comprises a branching group derived from lysine, e.g., a branching group of the following formula:

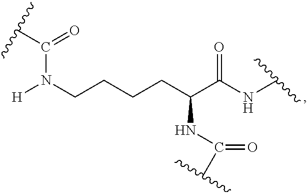 [SMILES: *NC(=O)[C@H](CCCCNC(*)=O)NC(*)=O]

or a salt thereof.

### Linking Moieties

As discussed above, the provided multi-chromophore dyes include a linking moiety. In some cases, the linking moiety comprises a functional group, such as a “chemoselective functional group”. Chemoselective functional groups are configured to selectively react with certain chemical moieties to form a new bond, e.g., a covalent bond. In some cases the chemoselective functional group is compatible with Click chemistry type reactions. The term “conjugation tag” refers to a group that includes a chemoselective functional group (e.g., as described herein) that can covalently link with a compatible functional group of a target molecule, after optional activation and/or deprotection. Any convenient conjugation tags may be utilized in the subject polymeric dyes in order to conjugate the dye to a target molecule of interest. In some embodiments, the conjugation tag includes a terminal functional group selected from an amino, a carboxylic acid or a derivative thereof, a thiol, a hydroxyl, a hydrazine, a hydrazide, a azide, an alkyne and a protein reactive group (e.g. amino-reactive, thiol-reactive, hydroxyl-reactive, imidazolyl-reactive or guanidinyl-reactive).

The linking moiety can be used to label the multi-chromophore dye by linking the multi-chromophore dye to another group, such as a conjugation tag configured to link to a target molecule, or to a specific binding member that specifically binds to a target molecule.

As used herein, the term “specific binding member” refers to one member of a pair of molecules which have binding specificity for one another. One member of the pair of molecules may have an area on its surface, or a cavity, which specifically binds to an area on the surface of, or a cavity in, the other member of the pair of molecules. Thus the members of the pair have the property of binding specifically to each other to produce a binding complex. In some embodiments, the affinity between specific binding members in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. In some embodiments, the specific binding members specifically bind with high avidity. By high avidity is meant that the binding member specifically binds with an apparent affinity characterized by an apparent K_{d} of 10×10⁻⁹ M or less, such as 1×10⁻⁹ M or less, 3×10⁻¹⁰ M or less, 1×10⁻¹⁰ M or less, 3×10⁻¹¹ M or less, 1×10⁻¹¹ M or less, 3×10⁻¹² M or less or 1×10⁻¹² M or less.

The specific binding member can be proteinaceous. As used herein, the term “proteinaceous” refers to a moiety that is composed of amino acid residues. A proteinaceous moiety can be a polypeptide. In certain cases, the proteinaceous specific binding member is an antibody. In certain embodiments, the proteinaceous specific binding member is an antibody fragment, e.g., a binding fragment of an antibody that specific binds to a polymeric dye. As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (l), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The term antibody is meant to include full length antibodies and may refer to a natural antibody from any organism, an engineered antibody, or an antibody generated recombinantly for experimental, therapeutic, or other purposes as further defined below.

Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions.

In certain embodiments, the specific binding member is a Fab fragment, a F(ab′)₂ fragment, a scFv, a diabody or a triabody. In certain embodiments, the specific binding member is an antibody. In some cases, the specific binding member is a murine antibody or binding fragment thereof. In certain instances, the specific binding member is a recombinant antibody or binding fragment thereof.

Any convenient methods and reagent may be adapted for use in the subject labeling methods in order to covalently link the conjugation tag to the target molecule. Methods of interest for labeling a target, include but are not limited to, those methods and reagents described by Hermanson, Bioconjugate Techniques, Third edition, Academic Press, 2013. The contacting step may be performed in an aqueous solution. In some instances, the conjugation tag includes an amino functional group and the target molecule includes an activated ester functional group, such as a NHS ester or sulfo-NHS ester, or vice versa. In certain instances, the conjugation tag includes a maleimide functional group and the target molecule includes a thiol functional group, or vice versa. In certain instances, the conjugation tag includes an alkyne (e.g., a cyclooctyne group) functional group and the target molecule includes an azide functional group, or vice versa, which can be conjugated via Click chemistry.

Any convenient target molecules may be selected for labeling utilizing the subject methods. Target molecules of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell, a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially. In some embodiments, the target molecule is a specific binding member (e.g., as described herein). In certain instances, the specific binding member is an antibody. In some instances, the specific binding member is an antibody fragment or binding derivative thereof. In some case, the antibody fragment or binding derivative thereof is selected from the group consisting of a Fab fragment, a F(ab′)₂ fragment, a scFv, a diabody and a triabody.

### Arrangment of Dyes, Polymeric Spacer Domains, and Linking Moiety

As discussed above, the multi-chromophore dyes of embodiments of the invention include two or more dyes each comprising water solubilizing groups, two or more polymeric spacer domains, and a linking moiety. The two or more dyes can be three or more dyes, such as four or more, five or more, six or more, or seven or more. The two or more polymeric spacer domains can be three or more polymeric spacer domains, such as four or more, five or more, six or more, seven or more, eight or more, or nine or more. Any suitable combination of the number of dyes and the number of polymeric spacer domains can be used, e.g., wherein the number of polymeric spacer domains is equal or greater than the number of dyes.

In some cases, the two or more polymeric spacer domains are part of a branched backbone, e.g., a non-conjugated branched backbone, joining the two or more dyes to the linking moiety. Each dye can have the same or different chemical structure. Each polymeric spacer domain can have the same or different chemical structure. In some cases the multi-chromophore dye has two dyes and three spacer domains, e.g., with the structure:

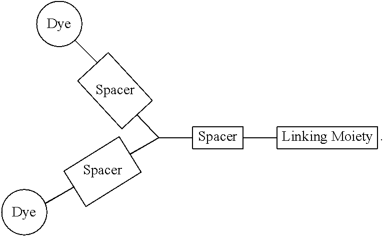

It is noted that the term “spacer” in the above diagram refers to the polymeric spacer domains described herein. In some embodiments, the multi-chromophore dye has three dyes and four polymeric spacer domains, e.g., with the structure:

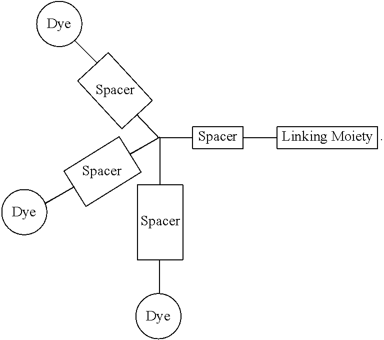

In some cases the multi-chromophore dye has four dyes and five or six polymeric spacer domains, e.g., with the structure:

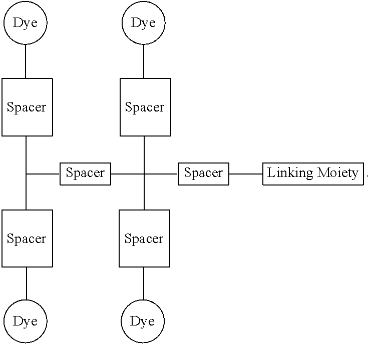

In such cases, the dyes and linking moiety can be referred to as pendant or side groups, wherein the polymeric spacer domains are part of the branched backbone. In some cases, the backbone of the multi-chromophore dye has one or more branching locations, such as two or more, three or more, or four or more.

In some embodiments, the multi-chromophore dye has a linear backbone, e.g. with the structure:

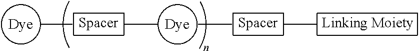

wherein n is a positive integer and, in some instances, ranges from 1 to 10, such as 2 to 8 and including 2 to 6.

### Methods

As summarized above, aspects of the invention include methods of evaluating a sample for the presence of a target analyte. Methods of embodiments of the invention include contacting the sample with a multi-chromophore dye specific binding member conjugate that specifically binds to the target analyte to produce a labeling composition, and assaying the labeling composition for the presence of a dye conjugated-target analyte binding complex to evaluate whether the target analyte is present in the sample. The multi-chromophore dye specific binding member conjugate comprises: a multi-chromophore dye as described herein and a specific binding member linked to the multi-chromophore dye.

As used herein, the term multi-chromophore dye specific binding member conjugate is used interchangeably with the term labeled specific binding member, the term labeling composition is used interchangeably with the term labeling composition contacted sample, and the term dye conjugated-target analyte binding complex is used interchangeably with the term labeled specific binding member-target analyte binding complex.

Any convenient method may be used to contact the sample with the multi-chromophore dye specific binding member conjugate. The solution may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. Various media are commercially available and may be used according to the nature of the target analyte, including dMEM, HBSS, dPBS, RPMI, Iscove's medium, etc., in some cases supplemented with fetal calf serum or human platelet lysate. The final components of the solution may be selected depending on the components of the sample which are included.

The temperature at which specific binding of the specific binding member of the conjugate to the target analyte takes place may vary, and in some instances may range from 5° C. to 50° C., such as from 10° C. to 40° C., 15° C. to 40° C., 20° C. to 40° C., e.g., 20 ° C., 25° C., 30° C., 35° C. or 37° C. (e.g., as described above). In some instances, the temperature at which specific binding takes place is selected to be compatible with the biological activity of the specific binding member and/or the target analyte. In certain instances, the temperature is 25° C., 30° C., 35° C. or 37° C. In certain cases, the specific binding member is an antibody or fragment thereof and the temperature at which specific binding takes place is room temperature (e.g., 25° C.), 30° C., 35° C. or 37° C. Any convenient incubation time for specific binding may be selected to allow for the formation of a desirable amount of binding complex, and in some instances, may be 1 minute (min) or more, such as 2 min or more, 10 min or more, 30 min or more, 1 hour or more, 2 hours or more, or even 6 hours or more.

Any convenient specific binding members may be utilized in the conjugate. Specific binding members of interest include, but are not limited to, those agents that specifically bind cell surface proteins of a variety of cell types, including but not limited to, stem cells, e.g., pluripotent stem cells, hematopoietic stem cells, T cells, T regulator cells, dendritic cells, B Cells, e.g., memory B cells, antigen specific B cells, granulocytes, leukemia cells, lymphoma cells, virus cells (e.g., HIV cells) NK cells, macrophages, monocytes, fibroblasts, epithelial cells, endothelial cells, and erythroid cells. Target cells of interest include cells that have a convenient cell surface marker or antigen that may be captured by a convenient specific binding member conjugate. In some embodiments, the target cell is selected from HIV containing cell, a Treg cell, an antigen-specific T -cell populations, tumor cells or hematopoetic progenitor cells (CD34+) from whole blood, bone marrow or cord blood. Any convenient cell surface proteins or cell markers may be targeted for specific binding to polymeric dye conjugates in the subject methods. In some embodiments, the target cell includes a cell surface marker selected from a cell receptor and a cell surface antigen. In some cases, the target cell may include a cell surface antigen such as CD11b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71.

Any convenient targets may be selected for evaluation utilizing the subject methods. Targets of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell, a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially. In some embodiments, the polymeric dye conjugates include an antibody or antibody fragment. Any convenient target analyte that specifically binds an antibody or antibody fragment of interest may be targeted in the subject methods.

In some embodiments, the target analyte is associated with a cell. In certain instances, the target analyte is a cell surface marker of the cell. In certain cases, the cell surface marker is selected from the group consisting of a cell receptor and a cell surface antigen. In some instances, the target analyte is an intracellular target, and the method further includes lysing the cell.

In some embodiments, the sample may include a heterogeneous cell population from which target cells are isolated. In some instances, the sample includes peripheral whole blood, peripheral whole blood in which erythrocytes have been lysed prior to cell isolation, cord blood, bone marrow, density gradient-purified peripheral blood mononuclear cells or homogenized tissue. In some cases, the sample includes hematopoetic progenitor cells (e.g., CD34+ cells) in whole blood, bone marrow or cord blood. In certain embodiments, the sample includes tumor cells in peripheral blood. In certain instances, the sample is a sample including (or suspected of including) viral cells (e.g., HIV).

The multi-chromophore dye specific binding member conjugates find use in the subject methods, e.g., for labeling a target cell, particle, target or analyte with a polymeric dye or polymeric tandem dye. For example, multi-chromophore dye specific binding member conjugate find use in labeling cells to be processed (e.g., detected, analyzed, and/or sorted) in a flow cytometer. The multi-chromophore dye specific binding member conjugate may include antibodies that specifically bind to, e.g., cell surface proteins of a variety of cell types (e.g., as described herein). The multi-chromophore dye specific binding member conjugate may be used to investigate a variety of biological (e.g., cellular) properties or processes such as cell cycle, cell proliferation, cell differentiation, DNA repair, T cell signaling, apoptosis, cell surface protein expression and/or presentation, and so forth. multi-chromophore dye specific binding member conjugate may be used in any application that includes (or may include) antibody-mediated labeling of a cell, particle or analyte.

Aspects of the method include assaying the labeling composition for the presence of a dye conjugated-target analyte binding complex to evaluate whether the target analyte is present in the sample. Once the sample has been contacted with the polymeric dye conjugate, any convenient methods may be utilized in assaying the labeling composition that is produced for the presence of a dye conjugated-target analyte binding complex. The dye conjugated-target analyte binding complex is the binding complex that is produced upon specific binding of the specific binding member of the conjugate to the target analyte, if present. Assaying the labeling composition can include detecting a fluorescent signal from the binding complex, if present. In some cases, the assaying includes a separating step where the target analyte, if present, is separated from the sample. A variety of methods can be utilized to separate a target analyte from a sample, e.g., via immobilization on a support. Assay methods of interest include, but are not limited to, any convenient methods and assay formats where pairs of specific binding members such as avidin-biotin or hapten-anti-hapten antibodies find use, are of interest. Methods and assay formats of interest that may be adapted for use with the subject compositions include, but are not limited to, flow cytometry methods, in-situ hybridization methods, enzyme-linked immunosorbent assays (ELISAs), western blot analysis, magnetic cell separation assays and fluorochrome purification chromatography.

In certain embodiments, the method further includes contacting the sample with a second specific binding member that specifically binds the target analyte. In certain instances, the second specific binding member is support bound. Any convenient supports may be utilized to immobilize a component of the subject methods (e.g., a second specific binding member). In certain instances, the support is a particle, such as a magnetic particle. In some instances, the second specific binding member and the polymeric dye conjugate produce a sandwich complex that may be isolated and detected, if present, using any convenient methods. In some embodiments, the method further includes flow cytometrically analyzing the dye conjugated-target analyte binding complex, i.e., a fluorescently labeled target analyte. Assaying for the presence of a dye conjugated-target analyte binding complex may provide assay results (e.g., qualitative or quantitative assay data) which can be used to evaluate whether the target analyte is present in the sample.

Any convenient supports may be utilized in the subject methods to immobilize any convenient component of the methods, e.g., multi-chromophore dye specific binding member conjugate, target, secondary specific binding member, etc. Supports of interest include, but are not limited to: solid substrates, where the substrate can have a variety of configurations, e.g., a sheet, bead, or other structure, such as a plate with wells; beads, polymers, particle, a fibrous mesh, hydrogels, porous matrix, a pin, a microarray surface, a chromatography support, and the like. In some instances, the support is selected from the group consisting of a particle, a planar solid substrate, a fibrous mesh, a hydrogel, a porous matrix, a pin, a microarray surface and a chromatography support. The support may be incorporated into a system that it provides for cell isolation assisted by any convenient methods, such as a manually-operated syringe, a centrifuge or an automated liquid handling system. In some cases, the support finds use in an automated liquid handling system for the high throughput isolation of cells, such as a flow cytometer.

In some embodiments of the method, the separating step includes applying an external magnetic field to immobilize a magnetic particle. Any convenient magnet may be used as a source of the external magnetic field (e.g., magnetic field gradient). In some cases, the external magnetic field is generated by a magnetic source, e.g., by a permanent magnet or electromagnet. In some cases, immobilizing the magnetic particles means the magnetic particles accumulate near the surface closest to the magnetic field gradient source, i.e. the magnet.

The separating may further include one or more optional washing steps to remove unbound material of the sample from the support. Any convenient washing methods may be used, e.g., washing the immobilized support with a biocompatible buffer which preserves the specific binding interaction of the polymeric dye and the specific binding member. Separation and optional washing of unbound material of the sample from the support provides for an enriched population of target cells where undesired cells and material may be removed.

In certain embodiments, the method further includes detecting the labeled target. Detecting the labeled target may include exciting the chromophore with one or more lasers and subsequently detecting fluorescence emission from the polymeric tandem dye using one or more optical detectors. Detection of the labeled target can be performed using any convenient instruments and methods, including but not limited to, flow cytometry, FACS systems, fluorescence microscopy; fluorescence, luminescence, ultraviolet, and/or visible light detection using a plate reader; high performance liquid chromatography (HPLC); and mass spectrometry. When using fluorescently labeled components in the methods and compositions of the present disclosure, it is recognized that different types of fluorescence detection systems can be used to practice the subject methods. In some cases, high throughput screening can be performed, e.g., systems that use 96 well or greater microtiter plates. A variety of methods of performing assays on fluorescent materials can be utilized, such as those methods described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In some cases, excitation radiation, from an excitation source having a first wavelength, passes through excitation optics. The excitation optics cause the excitation radiation to excite the sample. In response, fluorescently labeled targets in the sample emit radiation which has a wavelength that is different from the excitation wavelength. Collection optics then collect the emission from the sample. The device can include a temperature controller to maintain the sample at a specific temperature while it is being scanned. In certain instances, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

In some embodiments, the method of evaluating a sample for the presence of a target analyte further includes detecting fluorescence in a flow cytometer. In some embodiments, the method of evaluating a sample for the presence of a target analyte further includes imaging the labeling composition using fluorescence microscopy. Fluorescence microscopy imaging can be used to identify dye conjugated-target analyte binding complex in the labeling composition to evaluate whether the target analyte is present. Microscopy methods of interest that find use in the subject methods include laser scanning confocal microscopy.

Also provided are methods of labeling a target molecule. The subject dyes and tandem dyes find use in a variety of methods of labeling, separation, detection and/or analysis. In some embodiments, the method includes: contacting the target molecule with a polymeric tandem dye (e.g., as described herein) to produce a labeled target molecule, wherein the polymeric tandem dye includes a conjugation tag that covalently links to the target molecule. In some instances of the method, the polymeric dye member includes a chromophore according to any one of formulae (VA)-(VB) (e.g., as described herein), where one of G¹ and G² is a terminal group and the other of G¹ and G² is the conjugation tag.

The term “conjugation tag” refers to a group that includes a chemoselective functional group (e.g., as described herein) that can covalently link with a compatible functional group of a target molecule, after optional activation and/or deprotection. Any convenient conjugation tags may be utilized in the subject polymeric dyes in order to conjugate the dye to a target molecule of interest. In some embodiments, the conjugation tag includes a terminal functional group selected from an amino, a carboxylic acid or a derivative thereof, a thiol, a hydroxyl, a hydrazine, a hydrazide, a azide, an alkyne and a protein reactive group (e.g. amino-reactive, thiol-reactive, hydroxyl-reactive, imidazolyl-reactive or guanidinyl-reactive).

Any convenient methods and reagent may be adapted for use in the subject labeling methods in order to covalently link the conjugation tag to the target molecule. Methods of interest for labeling a target, include but are not limited to, those methods and reagents described by Hermanson, Bioconjugate Techniques, Third edition, Academic Press, 2013. The contacting step may be performed in an aqueous solution. In some instances, the conjugation tag includes an amino functional group and the target molecule includes an activated ester functional group, such as a NHS ester or sulfo-NHS ester, or vice versa. In certain instances, the conjugation tag includes a maleimide functional group and the target molecule includes a thiol functional group, or vice versa. In certain instances, the conjugation tag includes an alkyne (e.g., a cyclooctyne group) functional group and the target molecule includes an azide functional group, or vice versa, which can be conjugated via Click chemistry.

Any convenient target molecules may be selected for labeling utilizing the subject methods. Target molecules of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell, a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially. In some embodiments, the target molecule is a specific binding member (e.g., as described herein). In certain instances, the specific binding member is an antibody. In some instances, the specific binding member is an antibody fragment or binding derivative thereof. In some case, the antibody fragment or binding derivative thereof is selected from the group consisting of a Fab fragment, a F(ab′)₂ fragment, a scFv, a diabody and a triabody.

In some cases, the method includes a separating step where the labeled target molecule is separated from the reaction mixture, e.g., excess reagents or unlabeled target. A variety of methods may be utilized to separate a target from a sample, e.g., via immobilization on a support, precipitation, chromatography, and the like.

In some instances, the method further includes detecting and/or analyzing the labeled target molecule. In some instances, the method further includes fluorescently detecting the labeled target molecule. Any convenient methods may be utilized to detect and/or analyze the labeled target molecule in conjunction with the subject methods and compositions. Methods of analyzing a target of interest that find use in the subject methods, include but are not limited to, flow cytometry, fluorescence microscopy, in-situ hybridization, enzyme-linked immunosorbent assays (ELISAs), western blot analysis, magnetic cell separation assays and fluorochrome purification chromatography. Detection methods of interest include but are not limited to fluorescence spectroscopy, fluorescence microscopy, nucleic acid sequencing, fluorescence in-situ hybridization (FISH), protein mass spectroscopy, flow cytometry, and the like.

Detection may be achieved directly via the polymeric tandem dye, or indirectly by a secondary detection system. The latter may be based on any one or a combination of several different principles including, but not limited to, antibody labeled anti-species antibody and other forms of immunological or non-immunological bridging and signal amplification systems (e.g., biotin-streptavidin technology, protein-A and protein-G mediated technology, or nucleic acid probe/anti-nucleic acid probes, and the like). Suitable reporter molecules may be those known in the field of immunocytochemistry, molecular biology, light, fluorescence, and electron microscopy, cell immunophenotyping, cell sorting, flow cytometry, cell visualization, detection, enumeration, and/or signal output quantification. More than one antibody of specific and/or non-specific nature might be labeled and used simultaneously or sequentially to enhance target detection, identification, and/or analysis.

### Kits

Aspects of the invention further include kits for use in practicing the subject methods and compositions. The compositions of the invention can be included as reagents in kits either as starting materials or provided for use in, for example, the methodologies described above.

A kit can include a multi-chromophore dye (e.g., as described herein) and a container. Any convenient containers can be utilized, such as tubes, bottles, or wells in a multi-well strip or plate, a box, a bag, an insulated container, and the like. The subject kits can further include one or more components selected from a specific binding member, a specific binding member conjugate, a support bound specific binding member, a cell, a support, a biocompatible aqueous elution buffer, and instructions for use. In some embodiments of the kit, the linking moiety is covalently linked to a specific binding member. In some instances, the specific binding member is an antibody. In certain instances, the specific binding member is an antibody fragment or binding derivative thereof. In certain cases, the antibody fragment or binding derivative thereof is selected from the group consisting of a Fab fragment, a F(ab′)₂ fragment, a scFv, a diabody and a triabody.

In certain embodiments, the kit finds use in evaluating a sample for the presence of a target analyte, such as an intracellular target. As such, in some instances, the kit includes one or more components suitable for lysing cells. The one or more additional components of the kit may be provided in separate containers (e.g., separate tubes, bottles, or wells in a multi-well strip or plate).

In certain aspects, the kit further includes reagents for performing a flow cytometric assay. Reagents of interest include, but are not limited to, buffers for reconstitution and dilution, buffers for contacting a cell sample the chromophore, wash buffers, control cells, control beads, fluorescent beads for flow cytometer calibration and combinations thereof. The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The compositions of the kit may be provided in a liquid composition, such as any suitable buffer. Alternatively, the compositions of the kit may be provided in a dry composition (e.g., may be lyophilized), and the kit may optionally include one or more buffers for reconstituting the dry composition. In certain aspects, the kit may include aliquots of the compositions provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### Systems

Aspects of the invention further include systems for use in practicing the subject methods and compositions. A sample analysis system can include sample field of view or a flow channel loaded with a sample and a multi-chromophore dye specific binding member conjugate. In some embodiments, the system is a flow cytometric system including: a flow cytometer including a flow path; a composition in the flow path, wherein the composition includes: a sample; and a multi-chromophore dye specific binding member conjugate (e.g., as described herein).

In some embodiments, the system for analyzing a sample is a fluorescence microscopy system, including: a fluorescence microscope comprising a sample field of view; and a composition disposed in the sample field of view, wherein the composition comprises a sample; and a multi-chromophore dye specific binding member conjugate (e.g., as described herein).

In some instances of the systems, the multi-chromophore dye specific binding member conjugate includes: a tandem dye (e.g., as described herein) and a specific binding member that specifically binds a target analyte covalently linked to the chromophore.

In certain embodiments of the systems, the composition further includes a second specific binding member that is support bound and specifically binds the target analyte. In some cases, the support includes a magnetic particle. As such, in certain instances, the system may also include a controllable external paramagnetic field configured for application to an assay region of the flow channel.

The sample may include a cell. In some instances, the sample is a cell-containing biological sample. In some instances, the sample includes a multi-chromophore dye specific binding member conjugate specifically bound to a target cell. In certain instances, the target analyte that is specifically bound by the specific binding member is a cell surface marker of the cell. In certain cases, the cell surface marker is selected from a cell receptor and a cell surface antigen.

In certain aspects, the system may also include a light source configured to direct light to an assay region of the flow channel or sample field of view. The system may include a detector configured to receive a signal from an assay region of the flow channel or a sample field of view, wherein the signal is provided by the fluorescent composition. Optionally further, the sample analysis system may include one or more additional detectors and/or light sources for the detection of one or more additional signals.

In certain aspects, the system may further include computer-based systems configured to detect the presence of the fluorescent signal. A “computer-based system” refers to the hardware means, software means, and data storage means used to analyze the information of the present invention. The minimum hardware of the computer-based systems of the present invention includes a central processing unit (CPU), input means, output means, and data storage means. A skilled artisan can readily appreciate that any one of the currently available computer-based system are suitable for use in the subject systems. The data storage means may include any manufacture including a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g., word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

In certain aspects, the system includes a flow cytometer. Flow cytometers of interest include, but are not limited, to those devices described in U.S. Pat. Nos. 4,704,891; 4,727,029; 4,745,285; 4,867,908; 5,342,790; 5,620,842; 5,627,037; 5,701,012; 5,895,922; and 6,287,791; the disclosures of which are herein incorporated by reference.

Other systems may find use in practicing the subject methods. In certain aspects, the system may be a fluorimeter or microscope loaded with a sample having a fluorescent composition of any of the embodiments discussed herein. The fluorimeter or microscope may include a light source configured to direct light to the assay region of the flow channel or sample field of view. The fluorimeter or microscope may also include a detector configured to receive a signal from an assay region of the flow channel or field of view, wherein the signal is provided by the fluorescent composition.

### Utility

The multi-chromophore dyes and methods described herein may find use in a variety of applications, including diagnostic and research applications, in which the labeling, detection and/or analysis of a target of interest is desirable. Such applications include methodologies such as cytometry, microscopy, immunoassays (e.g. competitive or non-competitive), assessment of a free analyte, assessment of receptor bound ligand, and so forth. The compositions, system and methods described herein may be useful in analysis of any of a number of samples, including but not limited to, biological fluids, cell culture samples, and tissue samples. In certain aspects, the compositions, system and methods described herein may find use in methods where analytes are detected in a sample, if present, using fluorescent labels, such as in fluorescent activated cell sorting or analysis, immunoassays, immunostaining, and the like. In certain instances, the compositions and methods find use in applications where the evaluation of a sample for the presence of a target analyte is of interest.

In some cases, the methods and compositions find use in any assay format where the detection and/or analysis of a target from a sample is of interest, including but not limited to, flow cytometry, fluorescence microscopy, in-situ hybridization, enzyme-linked immunosorbent assays (ELISAs), western blot analysis, magnetic cell separation assays and fluorochrome purification chromatography. In certain instances, the methods and compositions find use in any application where the fluorescent labeling of a target molecule is of interest. The subject compositions may be adapted for use in any convenient applications where pairs of specific binding members find use, such as biotin-streptavidin and hapten-anti-hapten antibody.

The following examples are offered by way of illustration and not by way of limitation.

### Experimental

### EXAMPLE 1

### Design and Synthesis of New Multi-Chromophore Dyes

New multi-chromophore dyes were designed that have two or more dyes each having water solubilizing groups. The multi-chromophore dyes also had two or more polymeric spacer domains and a linking moiety.

One multi-chromophore dye was designed to have the following stucture. In particular, the multi-chromophore dye had a branched backbone joining the dyes and linking moiety, and the branched backbone had three polymeric spacer domains as shown.

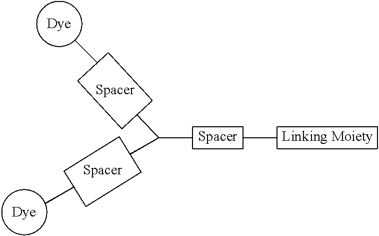

One example of such a multi-chromophore dye is shown below.

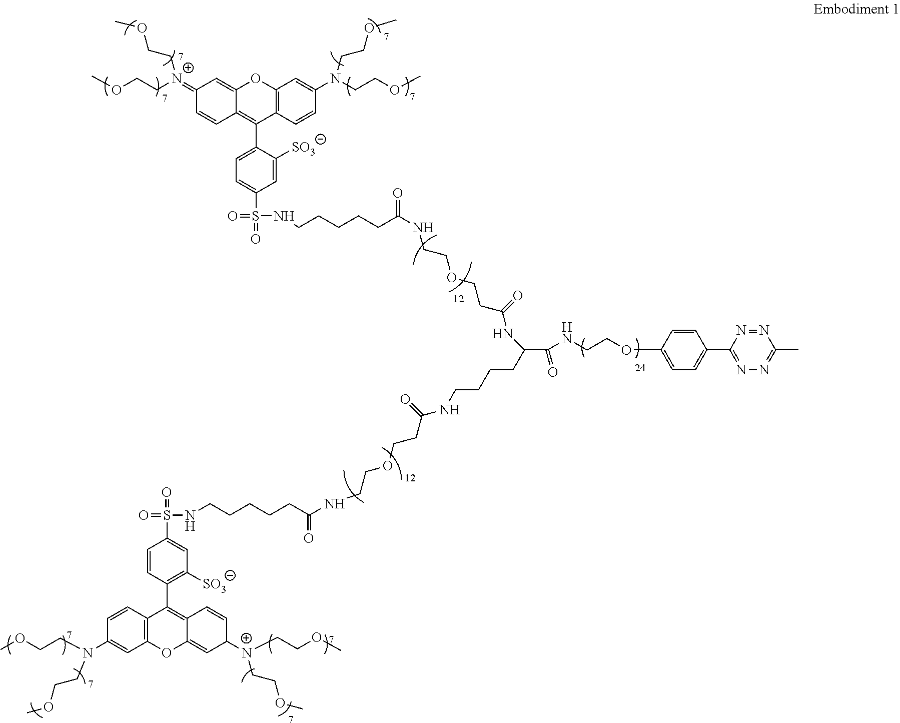

The central branching section of the multi-chromophore dye can be constructed by starting with a lysine molecule, which has the chemical structure shown below. The two amino groups and the one hydroxyl group can be used as the attachment points for the three groups of the multi-chromophore dye. For example, the amino groups can be reacted with carboxylic acids to give amide groups, and the carboxylic acid can be reacted an amino group to give the third amide group. The X¹, X², and X³ groups can correspond to the sections of the multi-chromophore dye shown above. The carboxylic acid groups can optionally be activated to increase the yield and ease of nucleophilic attack, such as by using thionyl chloride to give an acid chloride, or with formamide activation (e.g. Huy et al., “Formamide catalyzed activation of carboxylic acids—versatile and cost-efficient amidation and esterification”, Chemical Science, 2019, 31, doi: 10.1039/C9SC02126D).

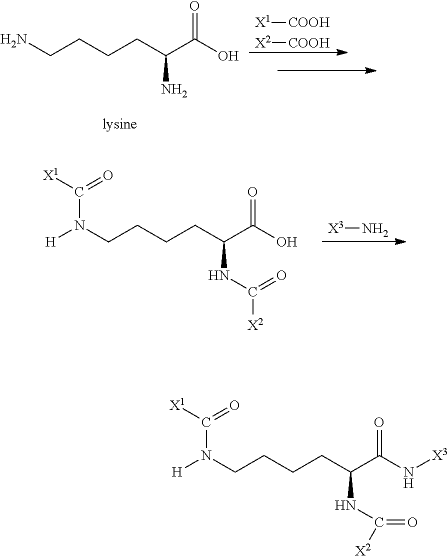

The dye of the exemplary multi-chromophore dye is a rhodamine-type dye wherein the phenyl ring is substituted with a sulfite group. Rhodamine dyes can be purchased from commercial suppliers or synthesized, such as according to the procedures of Mudd et al. (“A general synthetic route to isomerically pure functionalized rhodamine dyes”, Methods and Applications in Fluorescence, 2015, 3, doi:10.1088/2050-6120/3/4/045002). The nitrogen atoms of the rhodamine dye are substituted with polyethylene glycol groups (“Poly(ethylene glycol) Chemistry: Biotechnical and Biomedical Applications”, J. M. Harris, Ed., Plenum Press, New York, N.Y. (1992); and “Poly(ethylene glycol) Chemistry and Biological Applications”, J. M. Harris and S. Zalipsky, Eds., ACS (1997)). The phenyl ring of the rhodamine dye is also substitued with a sulfonamide group that is linked to an alkyl amide group, which is itself linked to a polymeric spacer domain, e.g., a polyethylene glycol group. The polyethylene glycol groups of the polymeric spacer domain and attached to the nitrogen of the rhodamine dye increase the water solubility of the multi-chromophore dyes.

The multi-chromophore dye also includes a phenyl ring and a tetrazine group, which can be used as a linking moiety. The linking moiety can be chemoselectively bonded to a specific binding member, such as an antibody, an antibody fragment, or a binding derivative thereof. The tetrazine can be used through chemoselective Click chemistry to react with an alkyne in a copper-catalyzed alkyne-azide cycloaddition click reaction (Presolski et al., “Copper-Catalyzed Azide-Alkyne Click Chemistry for Bioconjugation”, Current Protocols, 2011, 3, 4, 153, doi:10.1002/9780470559277.ch110148).

Additional exemplary multi-chromophore dyes with branched backbones are shown below. Variations include changing the number of repeat units in the polyethylene glycol groups, changing the identity of the dyes such as to other xanthene-based dyes or BODIPY dyes, and changing the identity of the water soluble groups of the dyes. Exemplary X groups for the chemical structures below include monovalent cations such as sodium or potassium as well as monovalant anions such as chloride for bromide. (Note that in the following list, there is intentionally no Embodiment 9 as Embodiment 9 is discussed in Example II below).

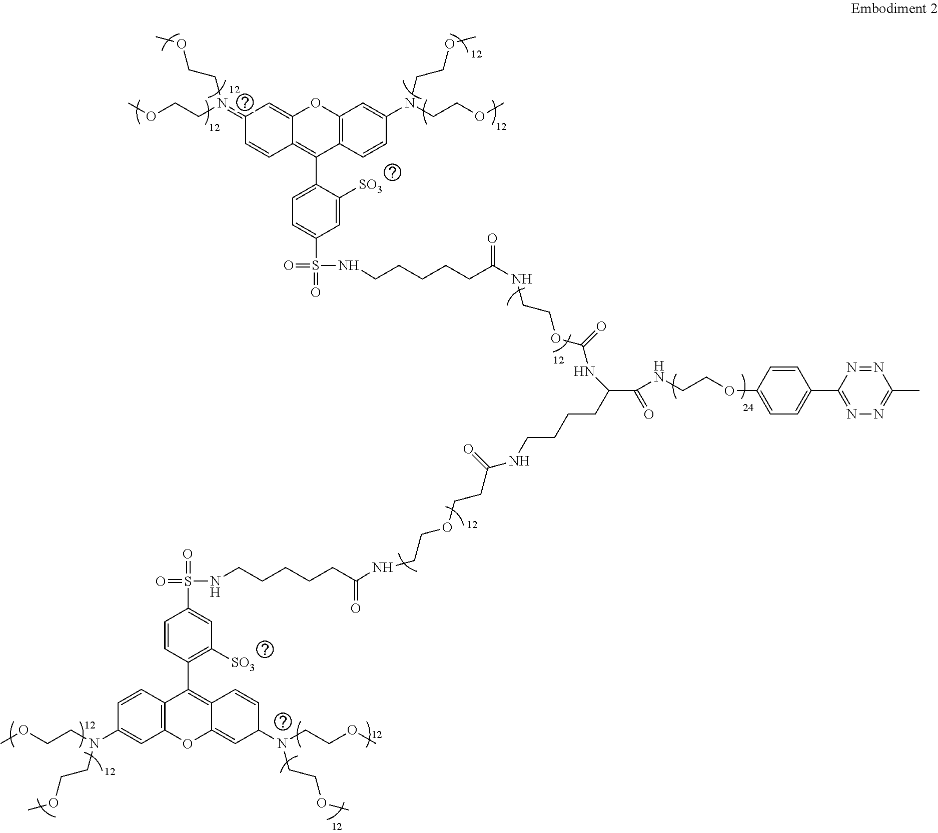

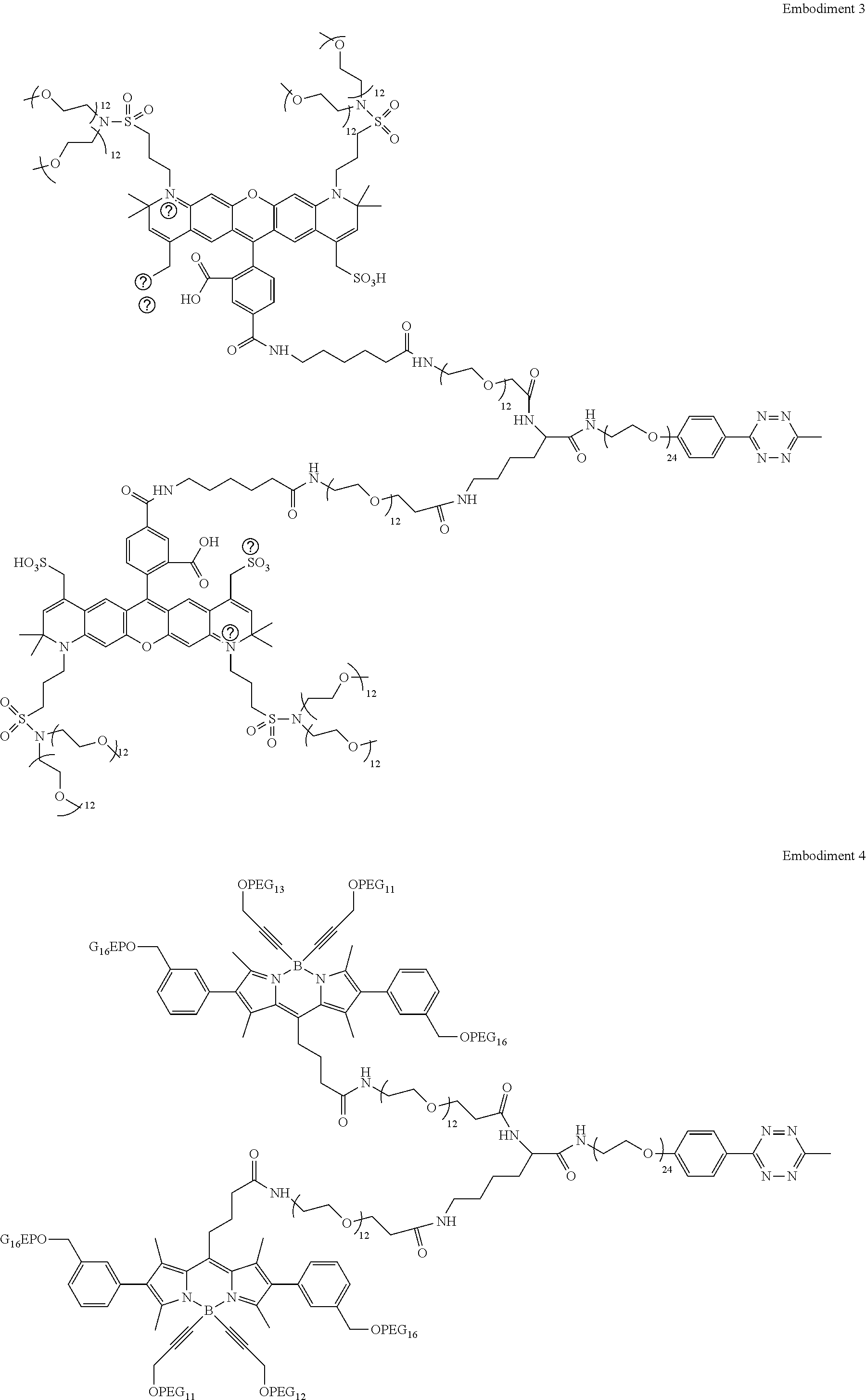

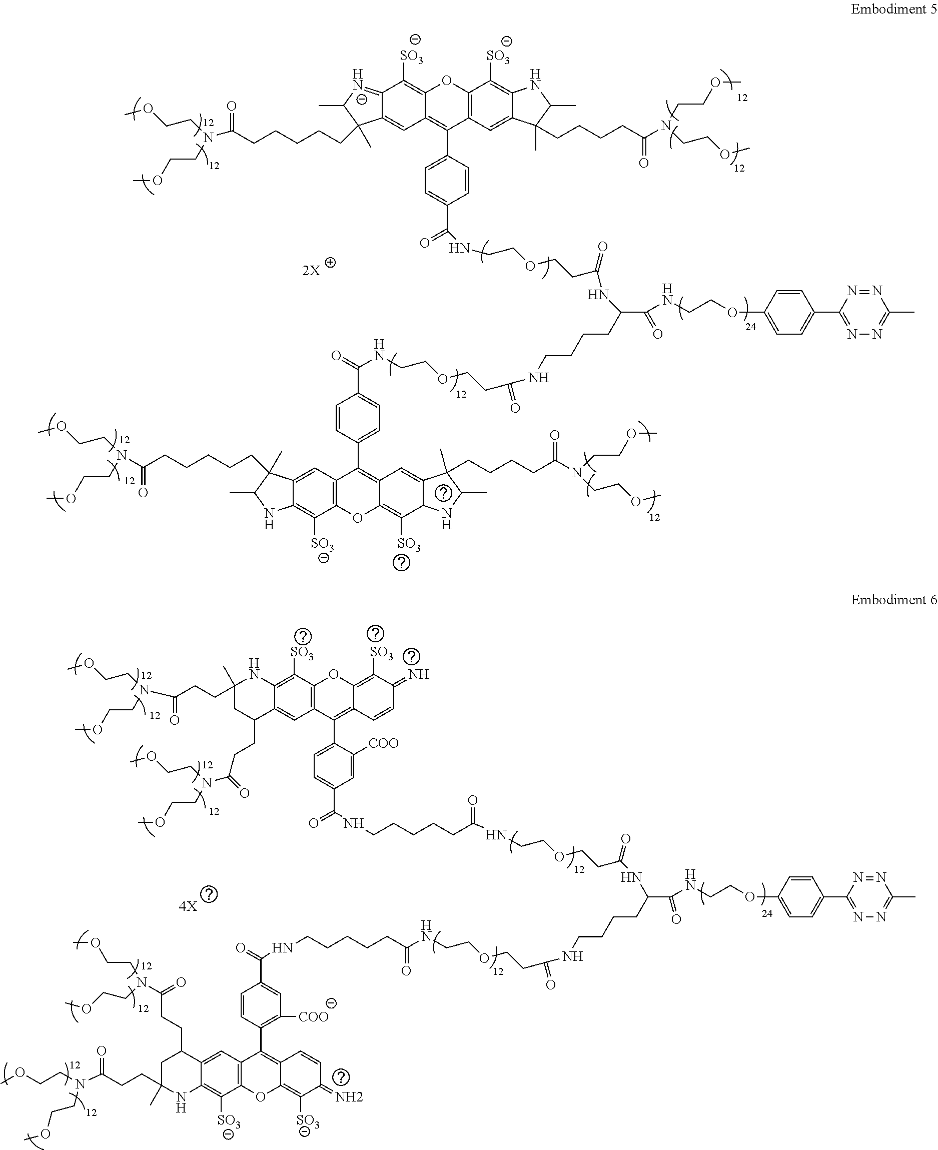

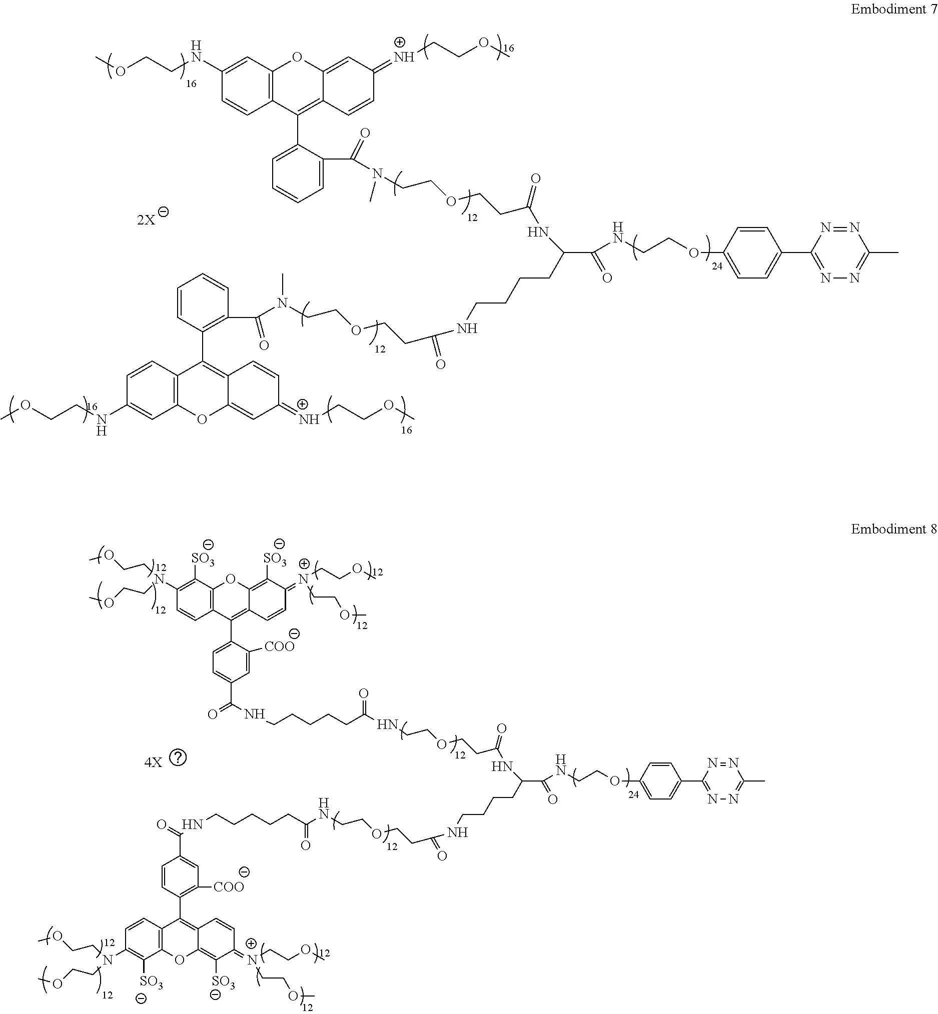

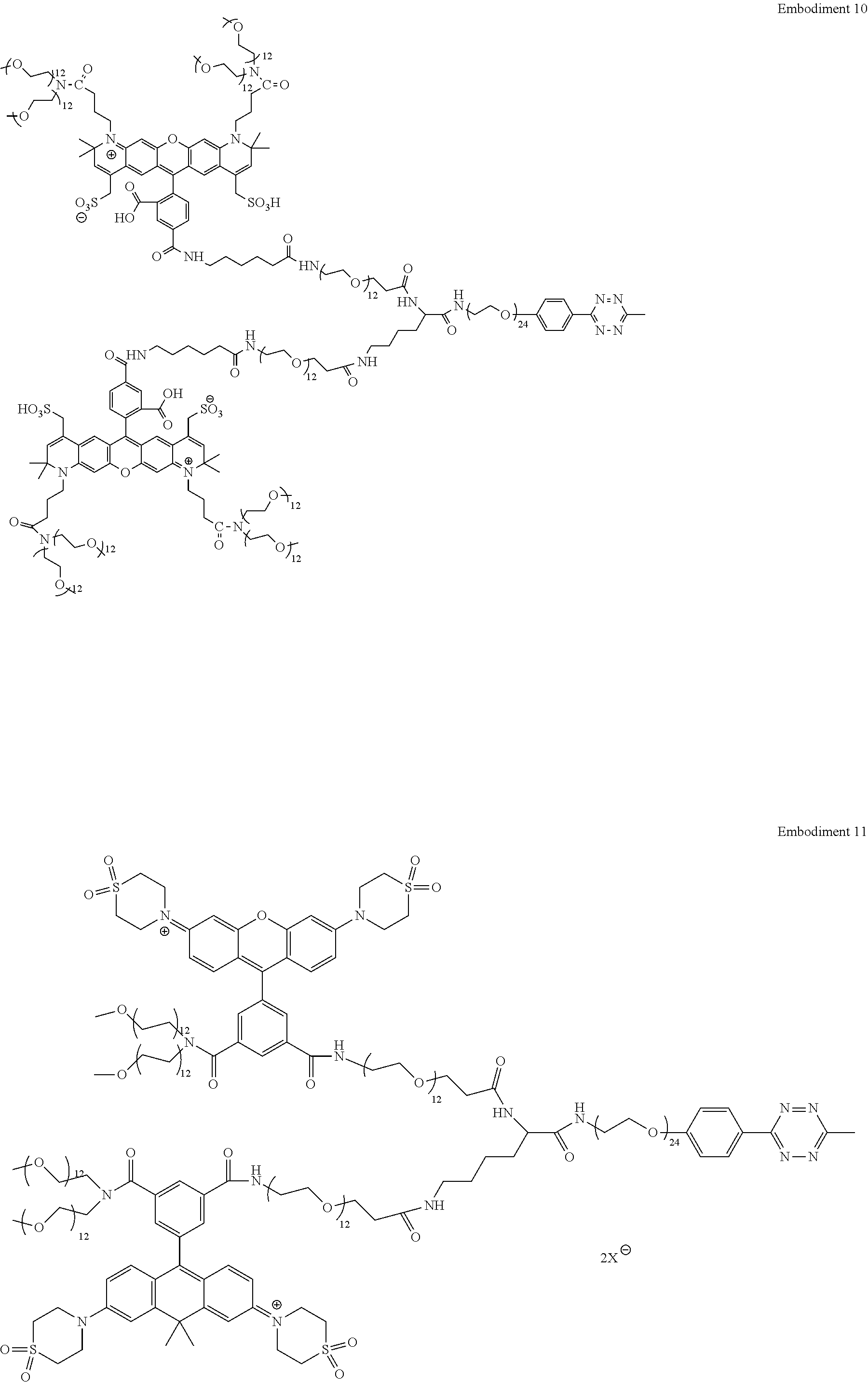

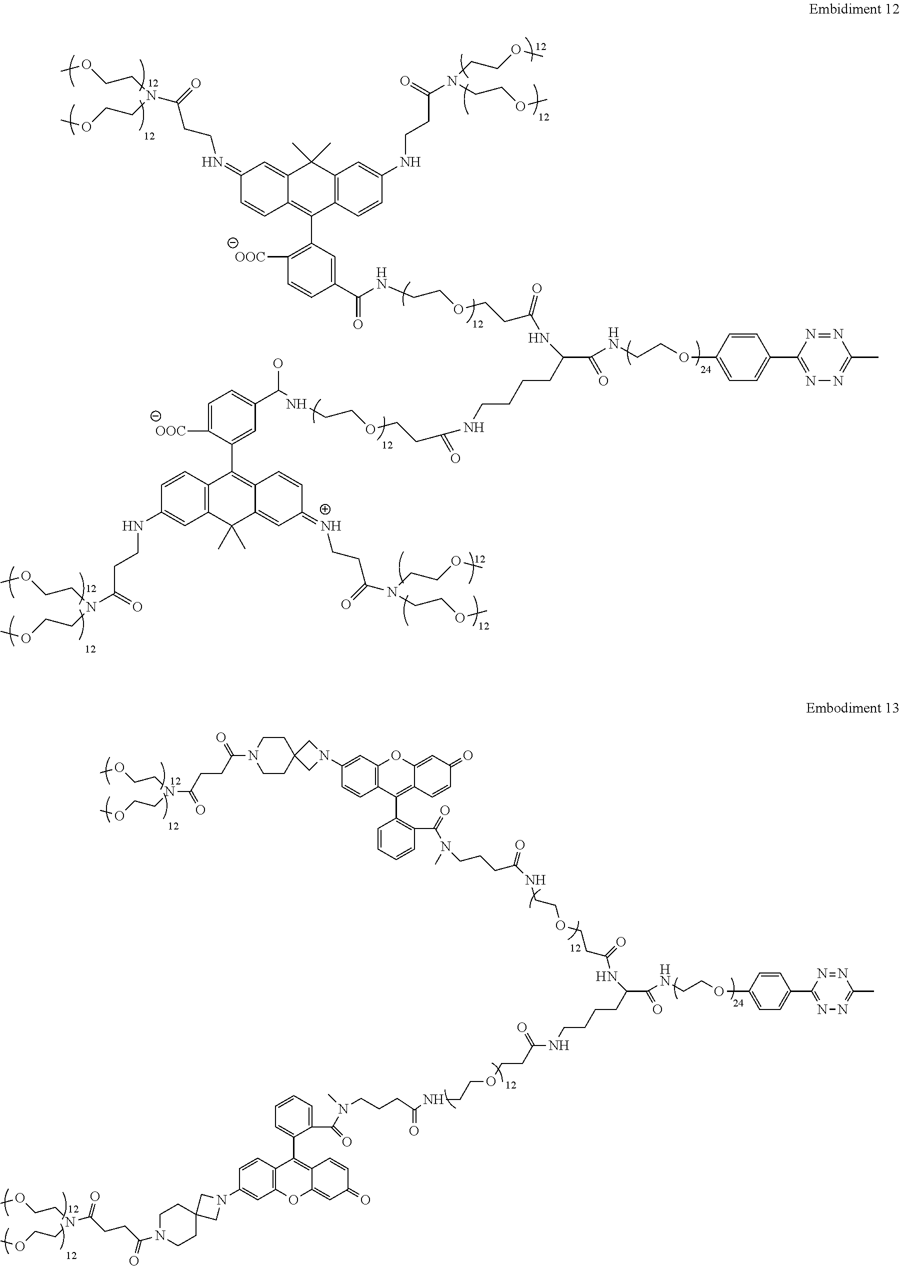

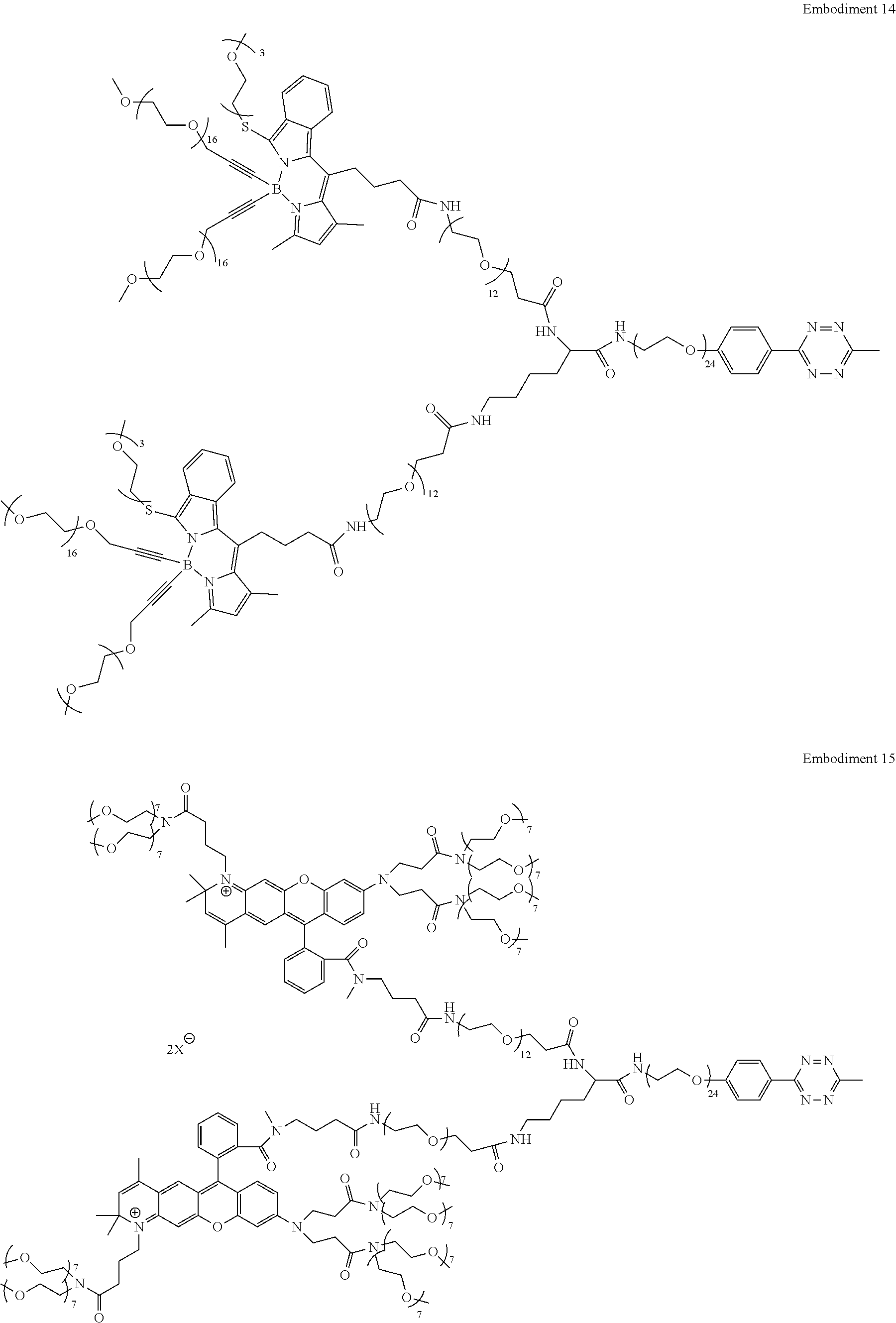

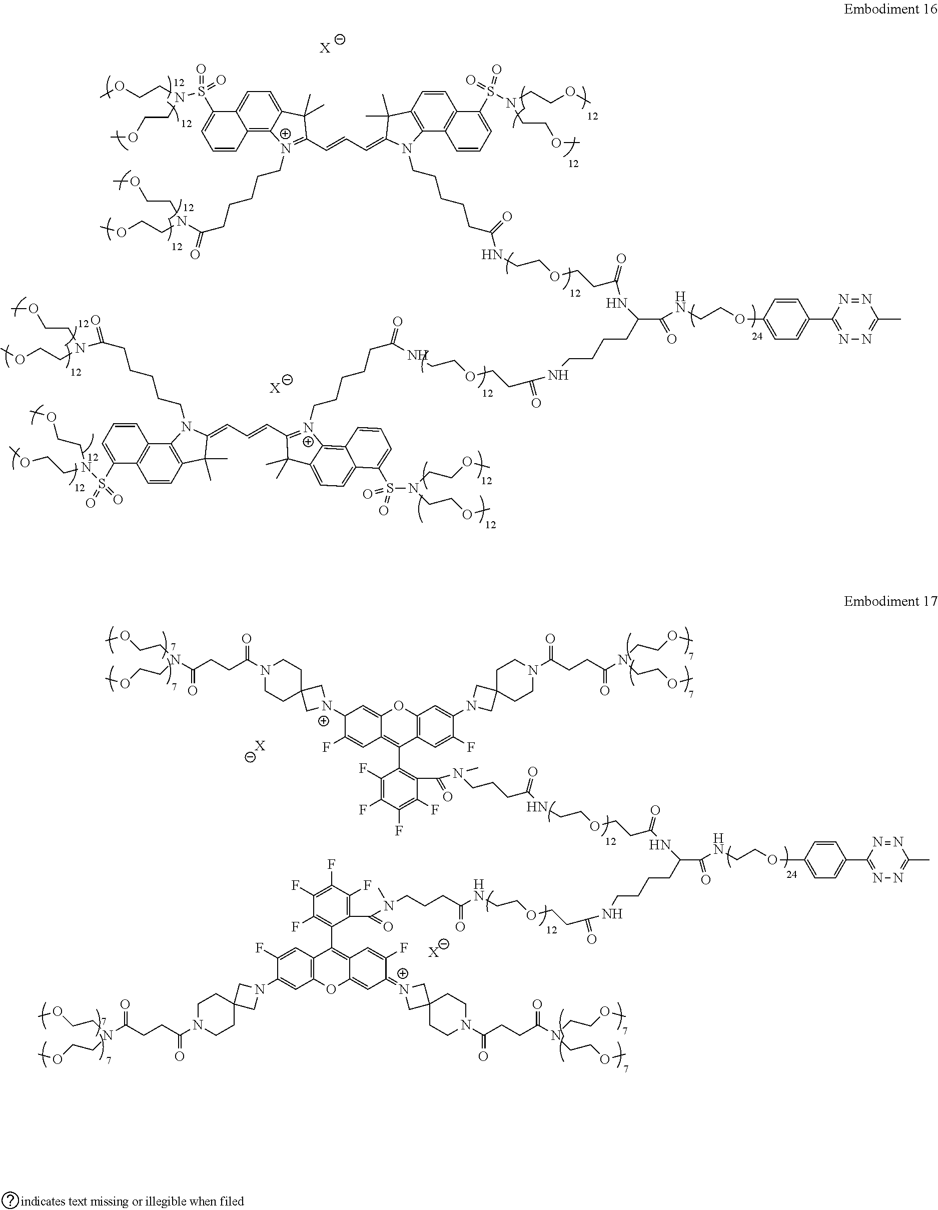

The multi-chromophore dyes can have molar extinction coefficients that are approximately double, triple, or quadruple of previous dyes, leading to greater brightness. The polyethylene glycol side chains located on the dyes, along with the polyethylene glycol sections of the polymeric spacer domains, can reduce self-quenching, thereby increasing the brightness of the multi-chromophore dyes. Hence, the overall increase in fluorescent intensity is significant, and the compositions can have high water solubility.

### EXAMPLE 2

### Optical Properties of Multi-Chromophore Dyes

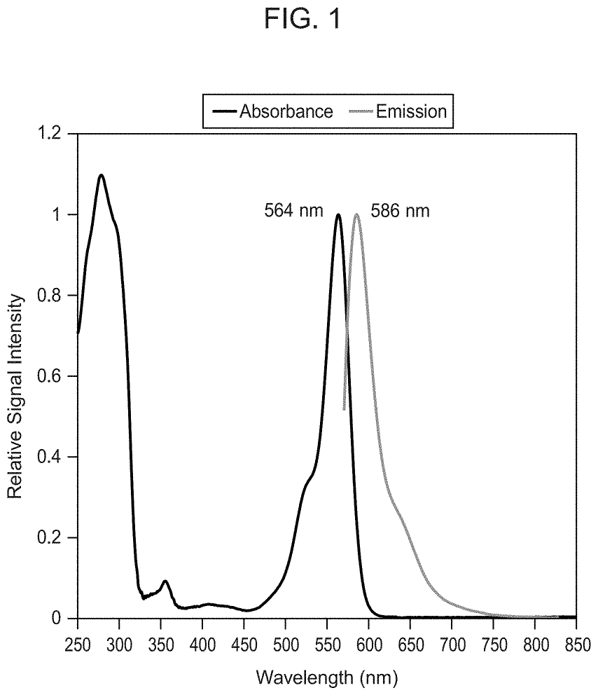

The absorption and emission spectrum of the Embodiment 2 multi-chromophore dye was measured (FIG. 1). The dye had an absorption maximum at 564 nm and an emission maximum at 586 nm, corresponding to a Stoke's shift of 22 nm.

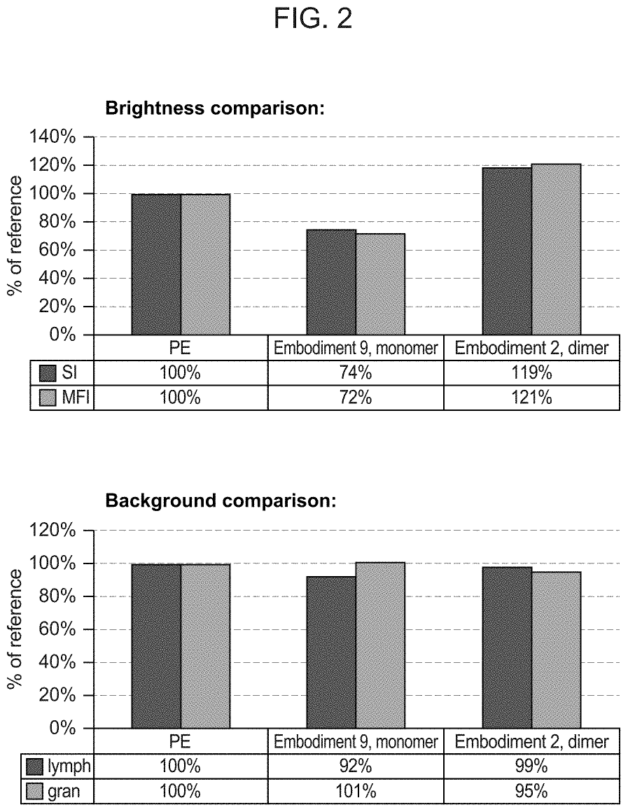

In addition, the brightness and background of multi-chromophore dyes of Embodiments 2 and 9 was also experimentally measured (FIG. 2). Whereas the Embodiment 9 multi-chromophore dye had a single dye connected the linking moiety through a single polymeric spacer domain, the Embodiment 2 multi-chromophore dye had two dyes connected the linking moiety through a branched backbone having three polymeric spacer domains.

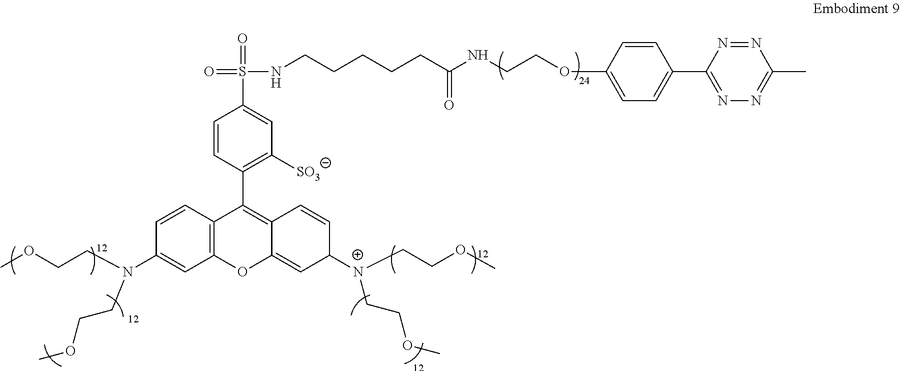

In addition, the Embodiment 9 structure that was experimentally measured was a monomeric species, whereas the Embodiment 2 species that was experimentally measured was a dimeric dye conjugate. As shown in FIG. 2, compared to PE the Embodiment 9 multi-chromophore dye had a lower brightness at 72% and 74%, whereas the Embodiment 2 multi-chromophore dye had a relative brightness of 119% and 121%. Hence, the Embodiment 2 multi-chromophore dye had significantly higher brightness than the PE and the Embodiment 9 composition.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

1. A multi-chromophore dye comprising:
two or more dyes each comprising a water solubilizing group;
two or more polymeric spacer domains; and
a linking moiety.

2. The multi-chromophore dye of claim 1, wherein the two or more polymeric spacer domains are part of a branched backbone joining the two or more dyes to the linking moiety.

3. The multi-chromophore dye of claim 2, wherein the multi-chromophore dye has the structure:

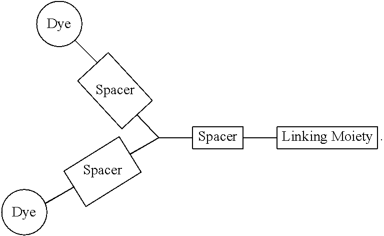

4. The multi-chromophore dye of claim 2, wherein the multi-chromophore dye has the structure:

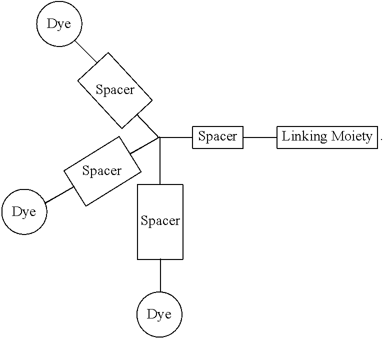

5. The multi-chromophore dye of claim 2, wherein the multi-chromophore dye has the structure:

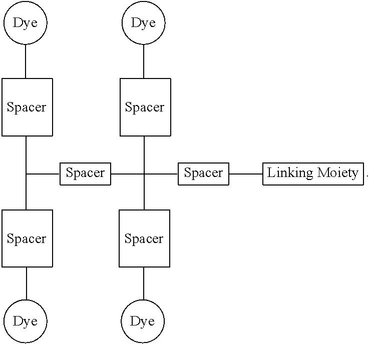

6. The multi-chromophore dye of claim 1, wherein the multi-chromophore dye has the structure: 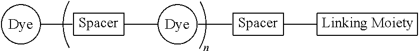
wherein n ranges from 1 to 100,000.

7. The multi-chromophore dye of claim 1, wherein the two or more polymeric spacer domains are each independently a poly(alkylene oxide) group.

8. The multi-chromophore dye of claim 7, wherein the poly(alkylene oxide) group is a poly(ethylene oxide) group.

9. The multi-chromophore dye of claim 7, wherein the poly(alkylene oxide) group has 4 to 50 alkylene oxide units.

10. The multi-chromophore dye of claim 9, wherein the poly(alkylene oxide) group has 8 to 30 alkylene oxide units.

11. The multi-chromophore dye of claim 1, wherein the linking moiety comprises a functional group.

12. The multi-chromophore dye of claim 11, wherein the functional group is a chemoselective functional group.

13. The multi-chromophore dye of any one of claims 12, wherein the linking moiety is bound to a specific binding member.

14. The multi-chromophore dye of claim 13, wherein the specific binding member is an antibody.

15. The multi-chromophore dye of claim 13, wherein the specific binding member is an antibody fragment or a binding derivative thereof.

16. The multi-chromophore dye of claim 15, wherein the antibody fragment or binding derivative thereof is selected from the group consisting of a Fab fragment, a F(ab′)₂ fragment, a scFv, a diabody and a triabody.

17. The multi-chromophore dye of claim 1, wherein each of the water solubilizing groups of the two or more dyes are independently a poly(alkylene oxide) group.

18. The multi-chromophore dye of claim 17, wherein each of the poly(alkylene oxide) groups is a poly(ethylene oxide) group.

19. The multi-chromophore dye of claim 17, wherein each of the poly(alkylene oxide) groups has 4 to 50 alkylene oxide units.

20. A method of evaluating a sample for presence of a target analyte, the method comprising:
(a) contacting the sample with a multi-chromophore dye specific binding member conjugate comprising:
(i) two or more dyes each comprising a water solubilizing group;
(ii) two or more polymeric spacer domains; and
(iii) a linking moiety bound to a specific binding member that specifically binds to the target analyte to produce a labeled composition; and
(b) assaying the labeled composition for the presence of a dye conjugated-target analyte binding complex to evaluate whether the target analyte is present in the sample.

21-40. (canceled)
